(19) United States
(12) Patent Application Publication
(10) Pub. No.: US 2024/0374705 A1
(43) Pub. Date: Nov. 14, 2024

(54) MUCOADHESIVE-PLGA NANOPARTICLES

(71) Applicants: PRESTIGE BIOPHARMA IDC CO., LTD, Busan (KR); PRESTIGE BIOPHARMA LIMITED, Singapore (SG)

(72) Inventors: Hee Dong HAN, Chungju-si Chungcheongbuk-do (KR); Tae In WI, Chungju-si Chungcheongbuk-do (KR)

(73) Assignees: PRESTIGE BIOPHARMA IDC CO., LTD, Busan (KR); PRESTIGE BIOPHARMA LIMITED, Singapore (SG)

(21) Appl. No.: 18/776,613

(22) Filed: Jul. 18, 2024

Related U.S. Application Data
(63) Continuation of application No. PCT/KR2023/001159, filed on Jan. 26, 2023.

(30) Foreign Application Priority Data
Jul. 26, 2022 (KR) 10-2022-0092488

(51) Int. Cl. A61K 39/12; A61K 9/00; A61K 39/00; A61K 47/59; A61K 47/69; A61P 35/00

(52) U.S. Cl. CPC A61K 39/12; A61K 9/006; A61K 47/59; A61K 47/6937; A61P 35/00; A61K 2039/542; A61K 2039/6093

(57) ABSTRACT

The present invention relates to a non-injectable drug delivery system using mucoadhesive nanoparticles, and to mucoadhesive nanoparticles and a preparation method therefor, the nanoparticles having a mucoadhesive polymer bound to the surface thereof, so that the deformation of nanoparticles caused by mucosal moisture and the like is prevented and adhesion to mucous membranes is strengthened, and thus the loss of nanoparticles is prevented. In addition, the present invention relates to a composition for maturing antigen-presenting cells, a composition for treating infectious disease and a composition for treating cancer, all of the compositions containing the mucoadhesive nanoparticles. Moreover, the present invention relates to mucoadhesive nanoparticles and an anticancer immunotherapy composition comprising same, the nanoparticles having immunoactive materials (such as antigens and adjuvants, etc.) for cancer treatment immunotherapy, based on antigen-presenting cells including dendritic cells (DC), loaded thereon.

### CROSS-REFERENCE TO RELATED APPLICATIONS

This application is a continuation of International Application No. PCT/KR2023/001159 filed on Jan. 26, 2023, which claims priority to Korean Patent Application No. 10-2022-0092488 filed on Jul. 26, 2022, the entire contents of which are herein incorporated by reference.

### TECHNICAL FIELD

The present invention relates to a non-injectable drug delivery system using mucoadhesive nanoparticles. Particularly, the present invention relates to mucoadhesive-poly (D,L-lactide-co-glycolide) (PLGA) nanoparticles in which a mucoadhesive polymer is bound to their surface, a method of preparing the same, a composition for inducing the maturation of antigen-presenting cells, which includes the mucoadhesive-PLGA nanoparticles, a composition for treating an infectious disease, and a composition for treating cancer.

### BACKGROUND ART

Immunotherapy is a method of treating cancer using the patient's own immune system, and is one of the main cancer treatment methods currently used, along with surgery, chemotherapy, and radiation therapy. Among these cancer treatment methods, immunotherapy is considered to be the safest and most effective with the fewest side effects.

Among immunotherapeutic agents used in immunotherapy, methods based on dendritic cells (DCs) are being actively studied. Dendritic cells are a representative type of antigen-presenting cells, and serve to enhance anti-cancer immunity by presenting tumor-specific antigens to help the tumor-specific activation of cytotoxic T cells (CD8+ T cells). To achieve the effective therapeutic efficacy of such dendritic cell-based immunotherapy, antigen delivery is the first key step. For this purpose, a syringe-based administration method is generally used, but syringe-based administration not only has side effects such as vascular weakness, vasoconstriction, and vascular occlusion, but also has the problem of frequently occurring serious symptoms such as needle phobia, and rejection of injections. Therefore, there is a need for alternative injection routes and delivery methods, which can effectively deliver drugs without using a syringe.

In this regard, research on non-injectable drug delivery treatments, which use a drug delivery method through a mucous membrane by spraying into the oral or nasal cavity is progressing, however due to the low mucosal adhesion of nanoparticles carrying such a drug, a large portion of the drug does not adhere to the mucous membrane and is lost, resulting in being delivered to the digestive system. As related art, drug delivery methods such as spraying or inhalation after a drug is loaded in nanoparticles are disclosed in Korean Unexamined Patent Publication No. 10-2021-0057072 and Korean Unexamined Patent Publication No. 10-2021-0124277, however the above-cited technologies are mainly characterized by technology of formulating a drug preparation to load a drug in nanoparticles or technology of delivering nanoparticles into the body via the lungs through nasal inhalation, and regarding the improvement in the mucosal adhesion of nanoparticles, nothing has been disclosed. In addition, most of the related art for cancer treatment using nanoparticles has the main technical feature of loading a chemical drug corresponding to an anticancer drug in a nanoparticle to directly deliver it to a target site (a cancer cell or tumor), and thus there is limitation that they are not suitable for being used in immunotherapy based on antigen-presenting cells, including dendritic cells.

### SUMMARY OF THE INVENTION

### Technical Problem

The present invention is mainly directed to providing, as a non-injectable drug delivery system using mucoadhesive nanoparticles, which has been developed to solve the conventional problems described above, mucoadhesive nanoparticles in which deformation of nanoparticles due to mucosal moisture is prevented and mucosal adhesion is strengthened to prevent their loss by binding a mucoadhesive polymer to the surface of nanoparticles, and a preparation method thereof. In addition, the present invention is directed to providing a composition for maturing antigen-presenting cells containing the mucoadhesive nanoparticles, a composition for treating an infectious disease, and a composition for treating cancer. In addition, the present invention is directed to providing mucoadhesive nanoparticles carrying immune activation materials (an antigen and an adjuvant, etc.) for cancer immunotherapy based on antigen-presenting cells, including dendritic cells (DCs), and a composition for cancer immunotherapy.

Specifically, the present invention is directed to providing mucoadhesive-poly(D,L-lactide-co-glycolide) (PLGA) nanoparticles in which a mucoadhesive polymer is bound to the surface of PLGA nanoparticles, and a preparation method thereof.

The present invention is also directed to providing a composition for inducing the maturation of antigen-presenting cells (APCs), which includes the mucoadhesive-PLGA nanoparticles as an active ingredient.

The present invention is also directed to providing a pharmaceutical composition for preventing or treating an infectious disease, which includes the mucoadhesive-PLGA nanoparticles as an active ingredient.

The present invention is also directed to providing a pharmaceutical composition for preventing or treating cancer, which includes the mucoadhesive-PLGA nanoparticles as an active ingredient.

The purpose of the present invention is not limited to the above description, but is provided for all cases where appropriate effects can be obtained by utilizing the present invention.

### Technical Solution

One aspect of the present invention provides mucoadhesive-PLGA nanoparticles in which a mucoadhesive polymer is bound to the surface of poly(D,L-lactide-co-glycolide) (PLGA) nanoparticles.

In addition, the mucoadhesive polymer of the present invention is selected from the group consisting of catechol (CAT), carrageenan, gelatin, pectin, and polyethylene glycol (PEG).

In addition, the mucoadhesive polymer of the present invention is CAT.

In addition, an antigen is contained in the nanoparticles.

In addition, the antigen is selected from the group consisting of a peptide, siRNA, and mRNA.

In addition, an adjuvant is further contained in the nanoparticles.

Another aspect of the present invention provides a composition for inducing the maturation of antigen-presenting cells (APCs), which includes the mucoadhesive-PLGA nanoparticles as an active ingredient.

In addition, the APCs are dendritic cells.

Still another aspect of the present invention provides a pharmaceutical composition for preventing or treating an infectious disease, which includes the mucoadhesive-PLGA nanoparticles as an active ingredient.

In addition, the pharmaceutical composition is for spraying into the oral cavity.

Yet another aspect of the present invention provides a pharmaceutical composition for preventing or treating cancer, which includes the mucoadhesive-PLGA nanoparticles as an active ingredient.

In addition, the pharmaceutical composition induces the maturation of APCs.

In addition, the pharmaceutical composition activates cytotoxic CD8+ T cells.

In addition, the pharmaceutical composition is for spraying into the oral cavity.

Yet another aspect of the present invention provides a method of preparing mucoadhesive-PLGA nanoparticles in which a mucoadhesive polymer is bound to the surface of poly (D,L-lactide-co-glycolide) (PLGA) nanoparticles, comprising: a) mixing an aqueous solution including an antigen and an adjuvant with an organic solution including PLGA; and b) mixing the mixture with a compound in which PVA-NH₂ in which an amino group is introduced into polyvinyl alcohol (PVA) and a mucoadhesive polymer in which a carboxyl group is introduced are chemically bonded to each other.

Yet another aspect of the present invention provides a method of preparing CAT-PLGA nanoparticles in which catechol (CAT) is bound to the surface of poly(D,L-lactide-co-glycolide) (PLGA) nanoparticles, comprising: a) mixing an aqueous solution including an antigen and an adjuvant with an organic solution including PLGA; and b) mixing the mixture with a compound (PVA-CAT) in which PVA-NH₂ in which an amino group is introduced into polyvinyl alcohol (PVA) and 3,4-dihyroxyhydrocinnamic acid (CAT-COOH) are chemically bonded to each other.

In one embodiment of the present invention, mucoadhesive-PLGA nanoparticles in which a mucoadhesive polymer is bound to the surface of poly(D,L-lactide-co-glycolide) (PLGA) nanoparticles are provided.

The “mucoadhesive polymer” used herein may be a compound with adhesive ability to a mucous membrane, especially mucosa in the body, such as oral mucosa or nasal mucosa, and include any polymer that is able to be applied to the body and has mucosal adhesion without limitation. Non-limiting examples of the mucoadhesive polymer may be catechol (CAT), carrageenan, gelatin, pectin, or polyethylene glycol (PEG). The mucoadhesive polymers have advantages of excellent adhesive ability to a mucous membrane, especially mucosa in the body, such as oral mucosa or nasal mucosa, and application to the body without side effects.

In the present invention, the mucoadhesive polymer may be CAT.

The “catechol (CAT)” used herein is a compound represented by Chemical Formula 1 below, and called 1,2-dihydroxybenzene.

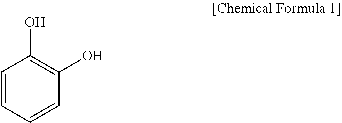

In the present invention, the CAT is bound to the surface of PLGA nanoparticles through chemical modification, thereby increasing the adhesion, adsorption, fixation, and deposition of the nanoparticles on a mucous membrane through physical entanglement, hydrogen bonding, hydrophobic bonding, or ionic interactions. In addition, CAT is biodegradable and non-toxic, so it may be used without side effects in vivo, and fixed to a functional amine group, thiol group, or imidazole group in a mucous membrane through chemical interactions on a mucous membrane containing moisture, such as oral mucosa or nasal mucosa.

The “poly(D,L-lactide-co-glycolide) (PLGA)” used herein is a copolymer of poly(lactic acid) (PLA) and poly(glycolic acid) (PGA), and represented by Chemical Formula 2 below.

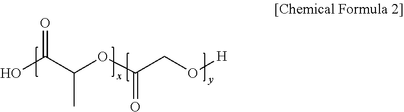

In Chemical Formula 2, x represents the number of lactic acid units, and y represents the number of glycolic acid units.

In the present invention, the ratio (x:y) of lactic acid:glycolic acid in PLGA is not particularly limited, and any ratio that can be used in the preparation of PLGA nanoparticles may be used. Non-limiting examples of the ratio of lactic acid: glycolic acid may be 90:10, 80:20, 70:30, 60:40, 50:50, 40:60, 30:70, 20:80, or 10:90, or more specifically, 30:70 to 70:30, and even more specifically, approximately 40:60 to 60:40. In one embodiment of the present invention, PLGA in which the ratio of lactic acid: glycolic acid is within approximately 50:50 was used, and when used in this range, as the in vivo decomposition rate of PLGA nanoparticles is more appropriately adjusted, the release rate of a drug loaded in the nanoparticles may be more appropriately adjusted, therefore, the above-mentioned ratio may be more suitable for preparing PLGA nanoparticles for in vivo drug delivery.

Since the mucoadhesive-PLGA nanoparticles of the present invention have a mucoadhesive polymer bound to the surface of the nanoparticles, deformation of nanoparticles due to moisture in a mucous membrane is prevented, and at the same time, the adhesion of the nanoparticle to the mucous membrane is strengthened to effectively adhere, fix, and deposit the nanoparticle on the mucous membrane, therefore, they have the advantage of preventing the loss of a drug loaded in the nanoparticles to effectively deliver it to cells.

In the present invention, the mucous membrane refers to a mucosa, especially a biological mucosa, to which the mucoadhesive-PLGA nanoparticles can come in contact and adhere, and non-limiting examples thereof may be oral mucosa or nasal mucosa.

In the present invention, the mucoadhesive-PLGA nanoparticles may contain an antigen therein.

The “antigen” used herein is an umbrella term for all materials that enter the body and cause immune responses, and generally, refers to any material that is recognized as a foreign material in the body and induces an immunoreactive state, and reacts with the immune cells or antibodies of a sensitized subject. The “antigen” used herein may be used collectively with the same meaning as the term “immunogen,” and may include molecules including one or more epitopes, which can stimulate a host immune system to produce a secretory, humoral, or cellular immune response specific for the antigen. In the present invention, non-limiting examples of the antigen may be a peptide (including a polypeptide and a protein), siRNA, or mRNA.

In one embodiment of the present invention, E7 protein was used as the antigen, and in this case, it may be loaded in the mucoadhesive-PLGA nanoparticles of the present invention with much higher efficiency and delivered into the body, and more effectively delivered to antigen-presenting cells to induce the maturation of the antigen-presenting cells and activate T cells, further, it can be effectively employed in cancer immunotherapy.

In the present invention, the mucoadhesive-PLGA nanoparticles may contain an adjuvant therein.

The “adjuvant” used herein means a helper or aid, refers to a material that positively affects the action of another material pharmacologically or immunologically, and is also called an immune enhancer or immune booster.

In the present invention, the type of adjuvant is not particularly limited, and any adjuvant used in the corresponding art or related fields may be used. Non-limiting examples of the adjuvant may include TLR3 adjuvants (Poly I:C, Poly I:CLC (hiltonol), PolyI:C12U (ampligen), and Poly I:C+CAF01 (CAF05)), TLR7/TLR8 adjuvants (Imiquimod (R-837), and resiquimod (R-848)), and TLR9 adjuvants (CpG ODN, and CpG ODN+MPL/QS21 (AS15)). In one embodiment of the present invention, poly I:C was used as the adjuvant, and in this case, it may be loaded, along with an antigen, in the mucoadhesive-PLGA nanoparticles of the present invention with excellent efficiency and delivered into the body, and thus can be effectively employed in cancer immunotherapy.

In another embodiment of the present invention, a composition for inducing the maturation of antigen-presenting cells (APCs), which includes the mucoadhesive-PLGA nanoparticles of the present invention as an active ingredient, is provided.

The “antigen-presenting cells (APCs)” used herein refer to cells that present an antigen-derived peptide fragment to T cells along with a major histocompatibility complex (MHC) molecule to activate the T cells after endocytosis of a protein antigen. Representative APCs include dendritic cells (DCs), B cells, and macrophages, and correspond to main immune cells that are responsible for cellular immunity in vivo. These cells serve as antigen-expressing cells, which present an antigen, which has entered the cells, on the surface after endocytosis and thus allow other cells (T cells, etc.) of the immune system to recognize the antigen.

Among the APCs, dendritic cells are known to be mainly present in tissue in contact with an external environment such as the skin, and the inner lining of the nose, lungs, stomach, or intestines, and particularly, cells in the skin are called Langerhans cells. Dendritic cells may be found in an immature state in the blood, and when activated, they are known to migrate to lymphoid organs and interact with T cells and B cells to initiate immune responses. In addition, the dendritic cells are known to extend projections called dendrites at a certain development stage.

Dendritic cells are derived from hemopoietic bone marrow progenitor cells, and these progenitor cells first change into immature dendritic cells, which are known to exhibit high endocytosis activity and T-cell activating ability. Immature dendritic cells constantly phagocytize pathogens such as surrounding viruses and bacteria, which is known to be mediated by a pattern recognition receptor (PRR), such as a toll-like receptor (TLR). TLRs recognize a specific chemical signature found on a subset of pathogens, and immature dendritic cells phagocytize the cell membrane of living autologous cells through a process called nibbling. When these immature dendritic cells come in contact with present antigens, they are activated into mature dendritic cells and migrate to the lymph nodes. Immature dendritic cells phagocytize pathogens and degrade their own proteins into small fragments when they mature, these fragments are presented on the cell surface using an MHC molecule, and at the same time, cell surface receptors acting as co-receptors in T-cell activation, such as cluster of differentiation (CD) 80, CD86, and CD40, increase. These cells may present antigens derived from pathogens, along with non-antigenic, specific co-stimulatory signals, thereby activating not only helper T cells and killer T cells, but also B cells.

Cytokines produced by dendritic cells vary depending on the type of cells. Lymphocytic dendritic cells may produce large amounts of type-1 interferon (IFN), which enables phagocytosis by recruiting more activated macrophages. Lymphocytic dendritic cells are involved in central and peripheral immune regulation, and myeloid dendritic cells are known to be involved in inducing immunity against foreign antigens or infections. Therefore, when dendritic cells do not function normally, autoimmune diseases such as diabetes, rheumatoid arthritis, and allergic hypersensitivity may occur, or normal immune responses to infectious diseases or cancer may not occur. As mentioned above, dendritic cells play an important role in enhancing the body's own immune function, so inducing the maturation of dendritic cells is an important task in cellular immunotherapy using dendritic cells.

The composition for inducing the maturation of APCs according to the present invention, which includes the mucoadhesive-PLGA nanoparticles as an active ingredient, exhibits an effect of inducing the maturation of immature APCs (e.g., dendritic cells) by effectively delivering immune activation materials (e.g., an antigen and an adjuvant, etc.) contained in the nanoparticles. Accordingly, when APCs mature, the increased expression of a surface protein marker may induce the activation of T cells, thereby increasing immune responses.

Specifically, mature APCs may allow MHC I-type and II-type antigens to be expressed at a higher level than immature APCs, and regulate CD (Cluster of Differentiation) 80+, CD83+, and CD86+. More MHC expression may lead to increased antigen density on the surface of APCs, and up-regulation of costimulatory molecules CD80 and CD86 may strengthen T cell-activating signals through their costimulatory molecular counterparts, such as CD28, on T cells.

The maturation of the APCs may be monitored by a known method in the art, and for example, a cell surface marker may be detected by an analytic method used in the art, such as flow cytometry or immunohistochemistry. In addition, the cells may be monitored through cytokine production analysis (e.g., ELISA or FACS).

In the present invention, the type of APC is not particularly limited, and non-limiting examples thereof may include dendritic cells, Langerhans cells, macrophages, mononuclear cells, or B cells. In one embodiment of the present invention, dendritic cells were targeted, and the dendritic cells are one of the most powerful APCs that serve as a bridge between the innate and adaptive immune systems and have the advantage of exhibiting higher immune activity.

In still another embodiment of the present invention, a pharmaceutical composition for preventing or treating an infectious disease, which includes the mucoadhesive-PLGA nanoparticles as an active ingredient, is provided.

The type of infectious disease in the present invention is not particularly limited, and the infectious disease may include diseases that are caused by various pathogens such as viruses, bacteria, and fungi.

In yet another embodiment of the present invention, a pharmaceutical composition for preventing or treating cancer, which includes the mucoadhesive-PLGA nanoparticles as an active ingredient, is provided.

In the present invention, the type of cancer (e.g., a specific disease name or disease site, etc.) is not particularly limited, and may include all types of cancer, whose symptoms can be improved, or prevented or treated by the maturation of APCs and the activation of immune cells such as T cells. The pharmaceutical composition for preventing or treating cancer according to the present invention, which includes the mucoadhesive-PLGA nanoparticles as an active ingredient, may not only treat cancer at a local site where it comes in direct contact with the composition, but also cancer or tumors, which occur in various organs, tissue, and cells in the body, through systemic circulation such as T cells activated by the pharmaceutical composition, and accordingly, T cell-mediated anticancer immunotherapy may be performed for systemic carcinoma. When the pharmaceutical composition of the present invention is prepared and used in an oral spray formulation, it can be more effective in treating, particularly oral cancer or head and neck cancer.

The pharmaceutical composition of the present invention may be prepared and used as a pharmaceutical composition for specific cancer/tumor-specific anticancer immunotherapy by loading an antigen or adjuvant specific to cancer/tumors to be targeted in the mucoadhesive-PLGA nanoparticles.

The pharmaceutical composition of the present invention may, particularly, effectively induce the maturation of APCs, and further promote the activation of T cells, for example, cytotoxic CD8+ T cells, and thus can be employed as an effective immunotherapy-based pharmaceutical composition in various ways.

The pharmaceutical composition of the present invention may be prepared in all formulations, which can be introduced into the body, and may be administered by all administration modes. In order to solve the problems of the conventional injection-based administration method, specifically, the pharmaceutical composition of the present invention was developed with nanoparticles with excellent mucosal adhesion, adsorption, fixing, and deposition ability, and accordingly, may be formulated to be easily administered to a mucous membrane, especially oral or nasal mucosa. In addition, it may be prepared as an oral spray formulation so that a drug can be delivered and absorbed more simply and effectively. A method of preparing the spray formulation is not particularly limited, and may be performed by a method used in the corresponding art or related fields.

In yet another embodiment of the present invention, there is provided a method of preparing mucoadhesive-PLGA nanoparticles in which a mucoadhesive polymer is bound to the surface of poly(D,L-lactide-co-glycolide) (PLGA) nanoparticles, comprising:
a) mixing an aqueous solution including an antigen and an adjuvant with an organic solution including PLGA; and
b) mixing the mixture with a compound in which PVA-NH₂ in which an amino group is introduced into polyvinyl alcohol (PVA), and a mucoadhesive polymer in which a carboxyl group (—COOH) is introduced are chemically bonded to each other.

The mucoadhesive-PLGA nanoparticles of the present invention may be prepared in the form of a water-in-oil-in-water (w/o/w) secondary emulsion by preparing a primary emulsion by dissolving an antigen and an adjuvant in a primary water phase and PLGA in an oil phase, i.e., an organic solvent, and mixing them, preparing a secondary emulsion by mixing an aqueous solution of PVA and an mucoadhesive polymer-binding compound, which is another secondary water phase, in the primary emulsion, and then evaporating the oil of the prepared secondary emulsion.

In yet another embodiment of the present invention, there is provided a method of preparing CAT-PLGA nanoparticles in which catechol (CAT) is bound to the surface of poly (D,L-lactide-co-glycolide) (PLGA) nanoparticles, comprising:
a) mixing an aqueous solution including an antigen and an adjuvant with an organic solution including PLGA; and
b) mixing the mixture with a compound (PVA-CAT) in which PVA-NH₂ in which an amino group is introduced into polyvinyl alcohol (PVA), and 3,4-dihyroxyhydrocinnamic acid (CAT-COOH) are chemically bonded to each other.

The CAT-PLGA nanoparticles of the present invention may be prepared in the form of a water-in-oil-in-water (w/o/w) secondary emulsion by preparing a primary emulsion by dissolving an antigen and an adjuvant in a primary water phase with PLGA in an oil phase, i.e., an organic solvent, and mixing them, preparing a secondary emulsion by mixing the primary emulsion with a PVA-CAT aqueous solution, which is a secondary water phase, and then evaporating the oil of the prepared secondary emulsion.

In the preparation of an emulsion formulation of PLGA nanoparticles, in the case of

PLGA nanoparticles prepared using general PVA as a secondary water phase, the nanoparticles may be deformed by moisture in a mucous membrane to lose a drug loaded therein, and due to very weak adhesion to a mucous membrane, the nanoparticles are not fixed to the mucous membrane and delivered to the digestive system. In contrast, in the present invention, mucoadhesive-PLGA nanoparticles in which a mucoadhesive polymer bound to the surface are prepared using a PVA-mucoadhesive polymer (e.g., PVA-CAT) in which a mucoadhesive polymer (e.g., CAT) and PVA are chemically bonded as a secondary water phase, thereby preventing deformation of nanoparticles due to moisture in a mucous membrane, and at the same time, strengthening the adhesion of the nanoparticle to the mucous membrane to effectively adhere, fix, and deposit the nanoparticle on the mucous membrane, therefore, they have the advantage of preventing the loss of a drug loaded in the nanoparticles to effectively deliver it to cells.

In one embodiment of the present invention, the reaction scheme illustrating the method and process of preparing PVA-CAT in which the CAT and PVA are chemically bonded is as follows.

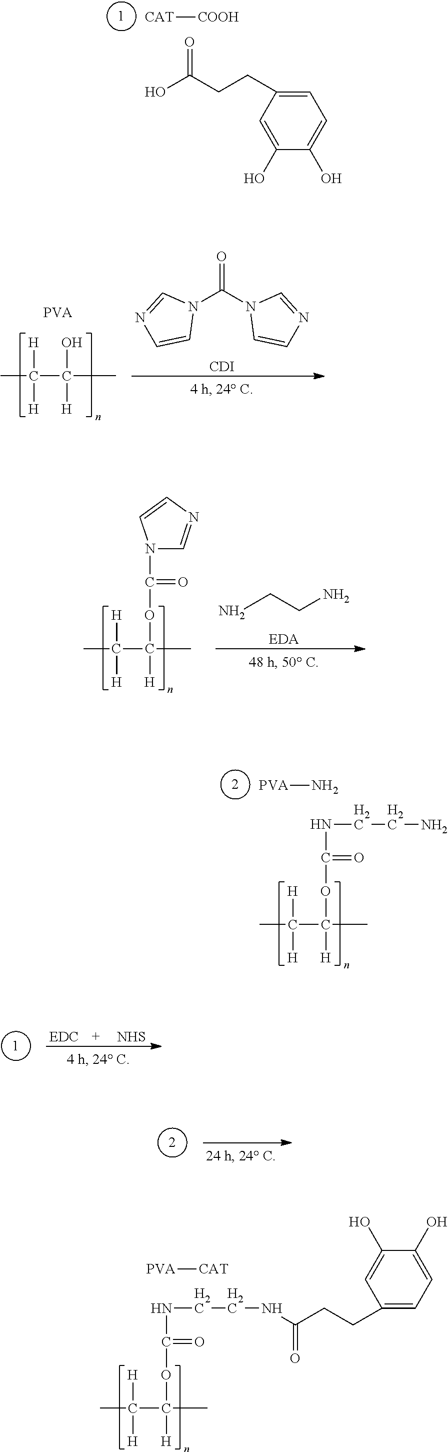

According to one embodiment of the present invention, the method of preparing PVA-CAT is as follows:

PVA-NH₂ was prepared by modifying PVA using carbonyldiimidazole (CDI) and ethylenediamine (EDA). Specifically, DMSO (60 mL) including CDI (74 mg) was added to PVA (400 mg), and then stirred at 24° C. for 4 hours. Subsequently, to remove unreacted reagents, the resulting solution was precipitated in butanol, CDI-activated PVA (CDI-PVA) was obtained, and then the CDI-PVA was dried in a vacuum oven. Subsequently, CDI-PVA (400 mg) and EDA (4 g) were dissolved in 100 mL of DMSO, stirred at 50° C. for 48 hours, and dried in a vacuum oven. In the second stage, CAT-COOH was conjugated with PVA-NH₂ using N-hydroxysuccinimide (NHS) and N-(3-dimethylaminopropyl)-N′-ethyl carbodiimide hydrochloride (EDC). Specifically, NHS (68 mg) and EDC (108 mg) were added to a CAT-COOH solution (37.3 mg/mL), and the mixture was stirred at 24° C. for 4 hours. Afterward, the PVA-NH₂ (400 mg) was added to the mixture, and additionally stirred at 24° C. for 24 hours. CAT-labeled PVA (PVA-CAT) was then separated using a dialysis membrane (cut off Mw000) and lyophilized, thereby obtaining PVA-CAT.

According to one embodiment of the present invention, a method of preparing CAT-PLGA nanoparticles according to the present invention using the PVA-CAT is as follows:

After dissolving 1 mg of E7 as an antigen and 2 mg of poly I:C as an adjuvant in 200 μL of deionized water, the mixed solution was mixed with 2 mL of a chloroform solution including PLGA (20 mg/ml) using a probe-type sonicator (SONICS, Newtown, CT, USA) under conditions of 4° C. and 30 seconds (6 pulses for 5 seconds at intervals of 3 seconds). The primary emulsion was sonicated with a secondary water phase (10 mL of 1.0% w/v PVA-CAT) at 4° C. for 5 minutes, thereby forming a secondary (w/o/w) emulsion. After completely evaporating chloroform using an evaporator for 10 minutes, CAT-PLGA (E7+poly I:C)-NPs were centrifuged at 15,800×g for 30 minutes, thereby preparing CAT-PLGA (E7+poly I:C)-NPs.

Terms not otherwise defined in the present invention are interpreted to have meanings conventionally used in the technical field. In addition, unless otherwise specified, the expression “or” used herein may be interpreted as a concept including “and.”

The scope of the present invention is not limited by the specific description disclosed in the present invention, and each description and embodiment disclosed in the present invention may be applied to other descriptions and embodiments. That is, all possible combinations of the various elements disclosed in the present invention are interpreted as falling within the scope of the present invention. In addition, those of ordinary skill in the art will be able to recognize or ascertain various equivalents to specific embodiments of the present invention through routine experimentation, and such equivalents should be construed as falling within the scope of the present invention.

### Benefit(s) of the Invention

The present invention relates to a non-injectable drug delivery system using mucoadhesive nanoparticles, and in the present invention, since a mucoadhesive polymer is bound to the surface of the nanoparticles, deformation of nanoparticles due to moisture in a mucous membrane is prevented, and at the same time, the adhesion of the nanoparticle to the mucous membrane is strengthened to effectively adhere, fix, and deposit the nanoparticle on the mucous membrane, therefore, the present invention has the advantage of preventing the loss of a drug loaded in the nanoparticles to effectively deliver it to cells. In addition, the present invention has the advantage in which, when antigens are loaded inside the nanoparticles, they can be effectively delivered in vivo or into cells, and used in various ways as a composition for inducing the maturation of APCs, a composition for treating an infectious disease, and a composition for treating cancer. In addition, the present invention has the advantage of being able to be employed in cancer immunotherapy through antigen-specific cytotoxic T cell (CD8+ T cell) activation by loading immune activating materials (e.g., an antigen and an adjuvant, etc.) for cancer immunotherapy based on APCs including dendritic cells (DCs) in the nanoparticles.

### BRIEF DESCRIPTION OF THE DRAWINGS

FIG. 1 is a schematic diagram illustrating the process of drug delivery and cancer treatment of spray-type CAT-PLGA-NPs of the present invention.

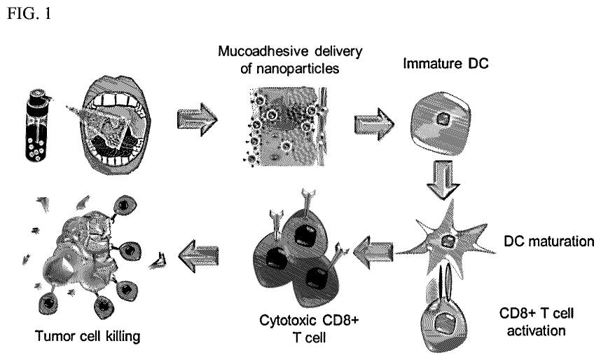

FIG. 2A shows a reaction scheme illustrating a PVA-CAT conjugation process by chemical modification in the present invention and FIG. 2B shows ¹H-NMR analysis results.

FIG. 3 shows the results of PVA-CAT conjugation analysis by FT-IR, wherein \textcircled{1} is a benzene aromatic ring of CAT-COOH, and \textcircled{2} is the amide I band of PVA-CAT.

FIGS. 4A-4D show the schematic diagrams, graphs, and images of the physicochemical properties of CAT-PLGA-NPs. Specifically, FIG. 4A is a schematic diagram illustrating the process of preparing CAT-PLGA (E7+poly I:C)-NPs. FIG. 4B shows the results of measuring the size and zeta potential of CAT-PLGA NPs through laser light scattering using a particle size analyzer. The loading efficiency of poly I:C and E7 onto CAT-PLGA-NPs was measured by NanoDrop (absorbance: 260 nm) and BCA protein analysis, respectively. FIG. 4C shows the results of measuring the size and zeta potential of CAT-PLGA NPs after spraying, and the loading efficiency of poly I:C and E7 in PLGA-NPs and CAT-PLGA-NPs after spraying. FIG. 4D shows the results of measuring the morphology of CAT-PLGA-NPs by FE-SEM (scale bar: 500 nm). FIG. 4E shows the results of measuring the cumulative release of E7 from CAT-PLGA (E7+poly I:C)-NPs under conditions of 37° C. and 50 RPM. FIG. 4F shows the results of measuring mucin adsorption to CAT PLGA-NPs at 263 nm using a UV-vis spectrophotometer. The data is expressed as mean±S.D (n=3) (*p<0.001).

FIG. 5 shows the results of measuring the size distribution of CAT-PLGA-NPs before and after spraying through laser scattering using a particle analyzer.

FIGS. 6A and 6B show the graphs and images of measuring the binding and intracellular delivery of CAT-PLGA-NPs to DCs, and the maturation of DCs induced by CAT-PLGA (E7+poly I:C)-NPs. Specifically, FIG. 6A shows the results of measuring the binding of CAT PLGA-NPs to DCs using flow cytometry, and FIG. 6B shows the results of measuring the intracellular delivery of CAT-PLGA NP to DCs using a confocal microscope (magnification: ×200, scale bar: 20 μm). Error bars represent SEM (*p<0.001).

FIGS. 7A and 7B show the graphs of measuring the maturation and cytokine production of DCs induced by CAT-PLGA (E7+poly I:C)-NPs. Specifically, FIG. 7A shows the results of measuring the levels of surface markers (CD40, CD80, and CD86) through flow cytometry. FIG. 7B shows the results of quantifying proinflammatory cytokines in culture supernatants of DCs cultured along with CAT-PLGA (E7+poly I:C)-NPs and DCs activating factors through ELISA. Error bars represent SEM (*p<0.001, **p<0.05).

FIG. 8 shows the results of monitoring the in-vivo fluorescent images of mice after spraying CAT-PLGA-NPs through IVIS. Error bars represent SEM (*p<0.01).

FIG. 9 shows the results of the adhesion of CAT-PLGA-NPs to murine oral mucosa (magnification: ×200, scale bar: 100 μm).

FIGS. 10A-10D show the therapeutic efficacy of CAT-PLGA-NPs in TC-1 tumor tongue models. Specifically, the treatment with PLGA (E7+poly I:C)-NPs and CAT-PLGA (E7+poly I:C)-NPs began three weeks after the injection of TC-1 tumor cells into the tongues of C57BL6 mice. PLGA (E7+poly I:C)-NPs and CAT-PLGA (E7+poly I:C)-NPs were sprayed once every week at 50 μg each of E7 and poly I:C. FIG. 10A shows the experimental schedule for treatment, FIG. 10B shows a tongue weight graph, FIG. 10C shows tongue and tumor weight images, and FIG. 10D shows a graph representing mouse weights. Error bars represent SEM (*p<0.001, **p<0.01, ***p<0.05).

FIG. 11 shows the results of measuring cytotoxic IFN-γ+CD8+ T cells in the tongue, mandibular lymphoid and spleen cells through flow cytometry. Error bars represent SEM (*p<0.001, **p<0.01, ***p<0.05).

FIG. 12 shows the results of H&E staining and immunohistochemical analyses for the tongue. Specifically, the immunohistochemical analysis of a cell proliferation marker (Ki67), and the measurement of microvessel density (MVD, CD31), and TUNEL and CD8+ T cell infiltration were performed using mouse tongue tissue (H&E scale bar: 500 μm), (scale bar: 25 μm). Error bars represent SEM (*p<0.001, **p<0.01, ***p<0.05).

### DETAILED DESCRIPTION

Hereinafter, the present invention will be described in further detail with reference to specific examples. However, these examples are merely provided to illustrate the present invention, and the scope of the present invention should not be construed as being limited in any way by these examples.

### Materials

Poly(D,L-lactide-co-glycolide) (PLGA) (Resomer® RG502H, a 50:50n monomer ratio, MW 10-12 kDa), polyvinyl alcohol(PVA, MW 9-10 kDa), 3,4-dihyroxyhydrocinnamic acid (CAT-COOH), carbonyldiimidazole (CDI), ethylenediamine (EDA), N-hydroxysuccinimide (NHS), N-(3-dimethylaminopropyl)-N′-ethyl carbodiimide hydrochloride (EDC), porcine stomach-derived type II mucin, and polyinosinic-polycytidylic acid sodium salt (poly I:C) were purchased from Sigma-Aldrich (St. Louis, MO, USA). Dimethyl sulfoxide (DMSO) was purchased from Biosesang (Bundang, Korea). The E7 peptide (TNYLFSPNGPIARAW) was purchased from Anygen (Gwangju, Korea). Fetal bovine serum (FBS) was purchased from Welgene (Gyeongsan, Korea). RPMI 1640 medium was purchased from Biowest (Nuaille, France). Hoechst 33342 was purchased from Invitrogen (Carlsbad, CA, USA). Cy5.5-NHS was purchased from Lumiprobe (Hunt Valley, MD, USA). FITC-labeled anti-mouse CD11c, and PE-labeled anti-mouse CD40, CD80, and CD86 were purchased from BioLegend (San Diego, CA, USA). FITC-labeled anti-mouse IFN-γ, and mouse TNF-α, IL-6 and IL-1β ELISA Ready-SET-Go kit were purchased from eBioscience (San Diego, CA, USA). APC-conjugated anti-CD8a was purchased from Invitrogen (Waltham, MA, USA). All of the above materials were analytical grade and used without further purification.

### EXAMPLES

### Example 1: Chemical Modification of PVA-Conjugated CAT

PVA-NH₂ was prepared by modifying PVA using CDI and EDA. Specifically, DMSO (60 mL) including CDI (74 mg) was added to PVA (400 mg), and stirred at 24° C. for 4 hours. Subsequently, to remove unreacted reagents, the resulting solution was precipitated in butanol, CDI-activated PVA (CDI-PVA) was obtained, and then the CDI-PVA was dried in a vacuum oven. Subsequently, CDI-PVA (400 mg) and EDA (4 g) were dissolved in 100 mL of DMSO, stirred at 50° C. for 48 hours, and then dried in a vacuum oven.

In the second stage, CAT-COOH was conjugated with PVA-NH₂ using NHS and EDC. Specifically, NHS (68 mg) and EDC (108 mg) were added to a CAT-COOH solution (37.3 mg/mL), and the mixture was stirred at 24° C. for 4 hours. Subsequently, PVA-NH₂ (400 mg) was added to the mixture, and additionally stirred at 24° C. for 24 hours. Afterward, CAT-labeled PVA (PVA-CAT) was separated using a dialysis membrane (cut off Mw000) and lyophilized, thereby obtaining PVA-CAT.

PVA-CAT formation was confirmed using ¹H-NMR (500 MHZ, HRMAS-FT NMR, Billerica, MA, USA) and Fourier transform infrared spectroscopy (FT-IR; Nicolet 5700, Thermo, Waltham, MA, USA).

### Example 2: Preparation Of Catechol-Conjugated PLGA Nanoparticles (CAT-PLGA-NPs)

PLGA (E7+poly I:C)-NPs refer to PLGA nanoparticles including E7 as an antigen and poly I:C as an adjuvant, and were prepared by a water-in-oil-in-water (w/o/w) evaporation method. Specifically, 1 mg of E7 and 2 mg of poly I:C were dissolved in 200 μL of deionized water, and then 2 mL of a chloroform solution including PLGA (20 mg/ml) was mixed using a probe-type sonicator (SONICS, Newtown, CT, USA) under conditions of 4° C. for 30 seconds (6 pulses for 5 seconds at intervals of 3 seconds). The primary emulsion was sonicated with a secondary water phase (10 mL of 1.0% w/v PVA) at 4°° C. for 5 minutes, thereby forming a secondary (w/o/w) emulsion. After completely evaporating the chloroform using an evaporator for 10 minutes, PLGA (E7+poly I:C)-NPs were centrifuged at 15,800×g for 30 minutes and washed three times, and stored at 4° C. until use. The preparation of CAT-PLGA (E7+poly I:C)-NPs was the same as the preparation procedure for PLGA (E7+poly I:C)-NPs, but PVA-CAT was used as a secondary water phase.

The size and zeta potential of CAT-PLGA (E7+poly I:C)-NPs were measured through dynamic light scattering using an electrophoretic light scattering photometer (SZ-100, HORIBA, Kyoto, Japan). The loading efficiency of E7 was measured using a BCA protein assay kit (Pierce Biotechnology, Rockford, IL, USA), and the loading efficiency of poly I:C was measured at 260 nm using a NanoDrop 1000 spectrophotometer (Thermo Fisher Scientific, Waltham, MA, USA). The morphologies of PLGA-NPs and CAT PLGA-NPs before and after spraying were confirmed using a field emission scanning electron microscope (FE-SEM; SU8000, HITACHI, Tokyo, Japan).

### Example 3: E7 Release From CAT-PLGA (E7+poly I:C)-NPs

CAT-PLGA (E7+poly I:C)-NP was added to a microtube, and then put into a water bath for a predetermined time. Afterward, the microtube was centrifuged at 15,800×g for 60 minutes to collect a supernatant, and the cumulative release of E7 from CAT-PLGA (E7+poly I:C)-NPs was measured. The amount of released E7 was measured using a BCA protein assay kit.

### Example 4: Mucin Adsorption Of CAT-PLGA-Nps

To confirm the mucin adsorption to CAT-PLGA-NPs, a mixture of mucin and CAT-PLGA-NPs was prepared at various mixing ratios (mucin: NP w/w, 1:0.25, 1:1, 1:4). The mixture was centrifuged at 15,800×g for 30 minutes, and then the supernatant was collected to measure non-adsorbed mucin at 263 nm using a UV-vis spectrophotometer.

### Example 5: Preparation Of Mice And Cell Line

Female C57BL/6 mice (5-to 6-week-old) were purchased from ORIENT (Gapyeong, Korea). All mice were maintained according to the protocol approved by the Animal Care Committee of the Konkuk University Veterinary Hospital (Ref. No.: KU20214) for the appropriate use and management of specific pathogen-free residential facilities at Konkuk University.

TC-1 cells (expressing HPV16-type HPV E6 and E7 proteins) were cultured in RPMI 1640 medium supplemented with 0.1% gentamycin and 10% fetal bovine serum (Biowest, Nuaille, France). DCs were obtained from the bone marrow of C57BL/6 mice, and cultured in RPMI 1640 medium supplemented with 0.1% gentamycin, 10% FBS, and 20 ng/mL of mice recombinant granulocyte-macrophage colony-stimulating factor (GM-CSF).

### Example 6: Binding And Intracellular Delivery Of CAT-PLGA-Nps

To confirm the binding and intracellular delivery of CAT-PLGA-NPs, fluorescent dye Cy5.5 was loaded into CAT-PLGA-NPs as a model drug. To measure the binding efficiency of CAT-PLGA-NPs, DCs were cultured with CAT-PLGA(Cy5.5)-NPs at 37° C. for 5 and 30 minutes, respectively. After culture, DCs were washed with PBS, stained with FITC-labeled anti-CD11c, and subjected to flow cytometry (BD FACSCalibur with CELLQuest software,

BD Biosciences, Franklin Lakes, NJ, USA). For confocal microscopy, DCs were cultured with CAT-PLGA(Cy5.5)-NPs at 37° C. for 30 minutes. Afterward, DCs were fixed with 4% paraformaldehyde (w/v) at 24° C. for 10 minutes, and stained with 1 μM Sytox® green (Life Technologies, Carlsbad, CA, USA) in PBS for 10 minutes. The intracellular delivery of CAT-PLGA(Cy5.5)-NPs in DCs were observed using a confocal microscope (LSM 710, Carl Zeiss, Oberkochen, Germany).

### Example 7: DC Maturation And Cytokine Production

DCs were cultured in a 6-well plate at a density of 5×10⁶ cells/well. A DC-only control, poly I:C (50 μg), CAT-PLGA-NPs, PLGA (E7+poly I:C)-NPs (50 μg each of E7 and poly I:C), and CAT-PLGA (E7+poly I:C)-NPs (50 μg each of E7 and poly I:C) were cultured for 30 minutes, and then a PLGA-NP-containing medium was removed. The DCs were additionally cultured for 24 hours, and stained with FITC-anti-CD11c, PE-anti-CD40, PE-anti-CD80, and PE-anti-CD86. DC maturation was measured using flow cytometry, and cytokines (TNF-α, IL-6, and IL-1β) secreted from the DCs were confirmed using a cytokine-specific ELISA kit (eBioscience, San Diego, CA, USA).

### Example 8: Adhesion Of CAT-PLGA-NP To Mouse Oral Mucosa

To evaluate the mucous adhesion of CAT-PLGA(Cy5.5)-NPs, CAT-PLGA(Cy5.5)-NPs was sprayed on the oral mucosa of C57BL/6 mice. The fluorescent signal of CAT-PLGA(Cy5.5)-NPs was monitored using an in vivo imaging system (IVIS, excitation: 630 nm, and emission: 710 nm). To further evaluate the adhesive effect of CAT-PLGA(Cy5.5)-NPs, immunohistochemical (IHC) analysis was performed using oral mucosal tissue. CAT-PLGA(Cy5.5)-NPs were sprayed on the oral mucosal tissue layer and after 30 minutes of incubation, the mucosal tissue was immediately washed twice with PBS. The tissue was fixed using an optimum cutting temperature compound (OCT; Tissue Tek, Torrance, CA, USA). To perform IHC analysis, tissue slides were stained with Hoechst 33342, and analyzed using a fluorescent microscope (BX61-32FDIC, Olympus, Tokyo, Japan). In addition, the tissue was counter-stained with hematoxylin and eosin (H&E, Leica Biosystems, Buffalo, IL, USA), and analyzed using a microscope (Eclipse NI, Nikon, Tokyo, Japan).

### Example 9: Therapeutic efficacy of CAT-PLGA-NPs

To generate tumors, TC-1 cells (4×10⁴ cells in 25 μL HBSS) were injected into the tongues of C57BL/6 mice (n=5 for each group). To evaluate therapeutic efficacy, vaccination began 3 days after tumor cells were injected into mice. Three groups of mice, (1) a control (negative) and (2) a control, (3) PLGA (E7+poly I:C)-NPs, and (4) CAT-PLGA (E7+poly I:C)-NPs (50 μg each of E7 and poly I:C), were vaccinated a total of three times once every week, and tongue weights and mouse weights were recorded. In addition, to confirm the activation of cytotoxic CD8+ T cells, the tongue, mandibular lymph node, and spleen cells were collected from each mouse. The cells isolated from the tissue were resuspended in 1 mL of RPMI 1640 containing FBS 10%, gentamycin 0.1%, and β-mercaptoethanol 1.0%, and cultured with GolgiPlug (BD Biosciences, San Diego, CA, USA) and E7(1 μg/mL) for 24 hours. Afterward, the cells were washed and stained with APC-anti-CD8a and FITC-anti-IFN-γ to confirm IFN-γ+CD8+ T cells through flow cytometry. H&E analysis, cell proliferation (anti Ki67, Abcam, Cambridge, UK), microvessel density (MVD, anti-CD31, Abcam Cambridge, UK), apoptosis (TUNEL, Trevigen, Gaithersburg, MD, USA), and the IHC analysis for a CD8+T cell population (anti-CD8, BioLegend, San Diego, CA, USA) were performed using tongue tissue isolated from the mice. The stained tissue was analyzed using bright-field microscopy and fluorescence microscopy. The analyses were recorded in random fields on each slide (5 random fields at ×400 magnification).

### Statistical Analysis

Differences in continuous variables were analyzed using a student's t-test to compare two groups, and differences between several groups were compared using ANOVA. All p values <0.05 in the examples were considered statistically significant.

### EXPERIMENTAL RESULTS

### Experimental Result 1: Induction of Dendritic Cell (DC)-Based Anticancer Immune Responses by Tumor-Specific Antigen Delivery Using Sprayable CAT-PLGA-Nps in Oral Cancer

The present inventors designed and prepared CAT-PLGA-NPs, which is a spray-type mucosal fixation-deposition type nanoparticle carrier, and by simply and easily delivering tumor-specific antigens to DCs in oral cancer patients who reject injections, DC-based immune responses were effectively induced.

### Experimental Result 2: Chemical Modification of PVA-Conjugated CAT

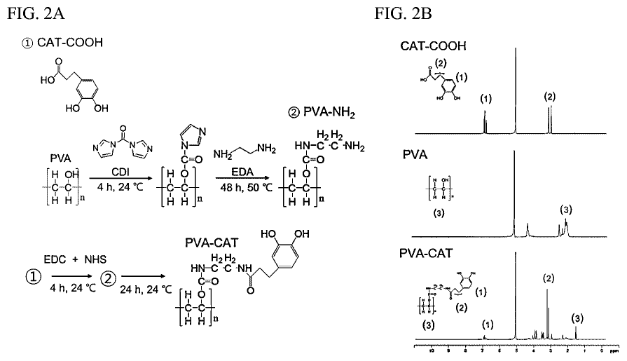

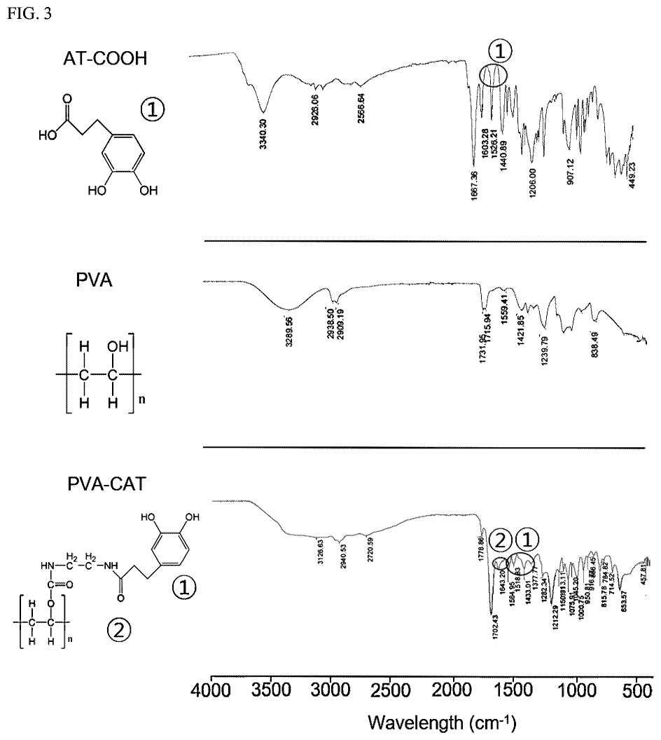

Prior to the preparation of CAT-PLGA-NPs, CAT-COOH and PVA-NH₂ were conjugated by chemical modification (FIG. 2A). The PVA-CAT conjugation was determined by ¹H-NMR spectra of (1) OH (aromatic C—OH) of CAT-COOH, (2) CH₂ (methylene) of CAT-COOH, and (3) CH₂ (methylene) of PVA (FIG. 2B). In addition, PVA-CAT was confirmed using FT-IR (FIG. 3). The absorption peaks of CAT-COOH and PVA-CAT between 1470 cm⁻¹ and 1622 cm⁻¹ result from an aromatic ring C═C vibrational mode (benzene aromatic ring). Particularly, the stretching C═O vibrational mode 1643 cm⁻¹ peak of the amide I band in PVA-CAT indicates that both CAT and PVA were well conjugated with COOH of CAT and NH₂ of PVA.

### Experimental Result 3: Physical Properties of CAT-PLGA-Nps

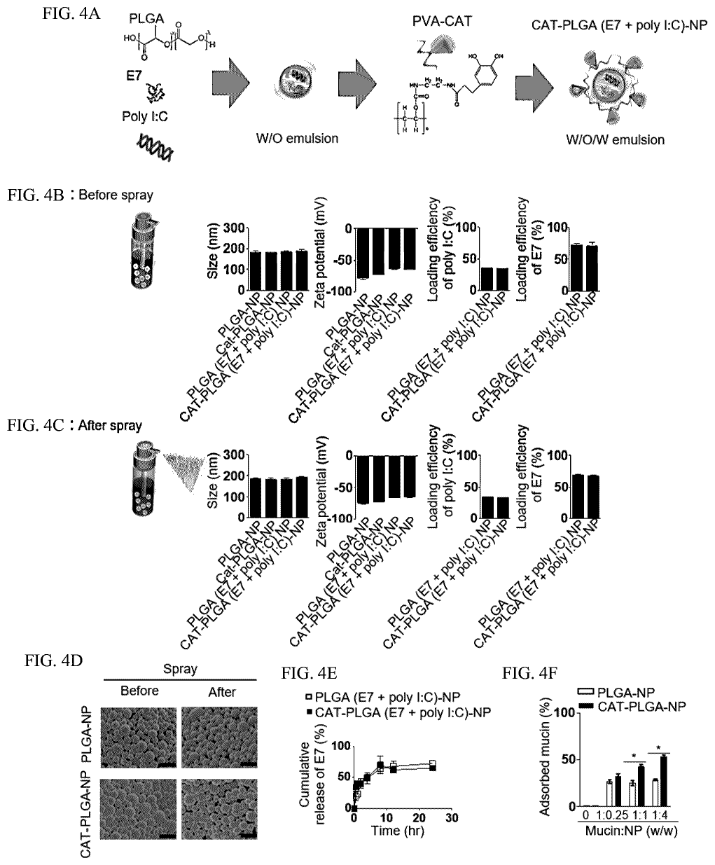

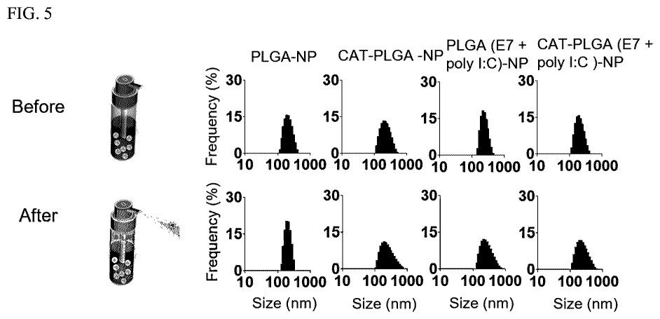

CAT-PLGA (E7+poly I:C)-NPs were prepared as shown in FIG. 4A. The sizes of PLGA-NPs and CAT-PLGA-NPs were approximately 200 nm (FIG. 4B). It was confirmed that the zeta potential was approximately −70 mV, and the loading efficiency of poly I:C and E7 was 35% (poly I:C) and 70% (E7), respectively (FIG. 4B). In addition, the sprayed PLGA-NPs and CAT-PLGA-NPs did not have difference compared to the conditions “before spraying” (FIG. 4C), and did not have difference in size distribution (FIG. 5). The morphologies of the PLGA-NPs and CAT-PLGA-NPs were detected by FE-SEM (FIG. 4D). The shape of CAT-PLGA-NPs before and after spraying was spherical, and there was no change in shape after spraying. These results suggest that CAT-PLGA-NPs are suitable as a spray-type formulation. Next, the release of E7 from CAT-PLGA-NPs was confirmed (FIG. 4E). The cumulative release patterns of E7 from the PLGA-NPs and CAT-PLGA-NPs were similar, and burst release was observed within 8 hours.

Mucin is an important glycoprotein in the mucosa and is responsible for mucosal structure. Therefore, the adsorption effect of CAT-PLGA-NPs was confirmed. Mucin was significantly well adsorbed to the surface of CAT-PLGA-NPs with an increasing amount of CAT-PLGA-NPs, compared to non-CAT-labeled PLGA-NPs (FIG. 4F).

### Experimental Result 4: Binding and Intracellular Delivery of CAT-PLGA-Nps

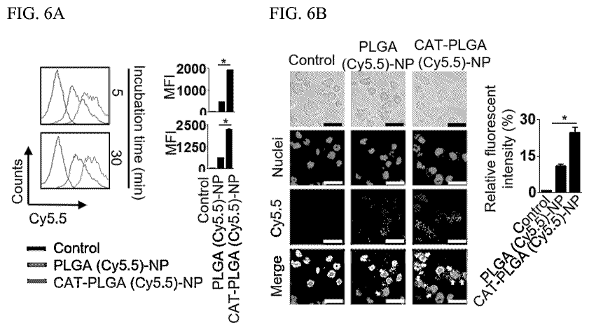

The binding of CAT-PLGA(Cy5.5)-NPs to DCs was measured using flow cytometry, and it was confirmed that CAT-PLGA(Cy5.5)-NPs exhibit higher binding efficiency than PLGA(Cy5.5)-NPs (FIG. 6A). In addition, the intracellular delivery of CAT-PLGA(Cy5.5)-NPs was measured using a confocal microscope. The relative fluorescence intensity of CAT-PLGA(Cy5.5)-NPs was confirmed to be higher than that of PLGA(Cy5.5)-NPs (FIG. 6B).

These results suggest that CAT-PLGA(Cy5.5)-NPs are more suitable for binding to DCs and intracellular delivery compared to PLGA(Cy5.5)-NPs by CAT labeling.

### Experimental Result 5: DC Maturation and Cytokine Production Induced by CAT-PLGA-Nps

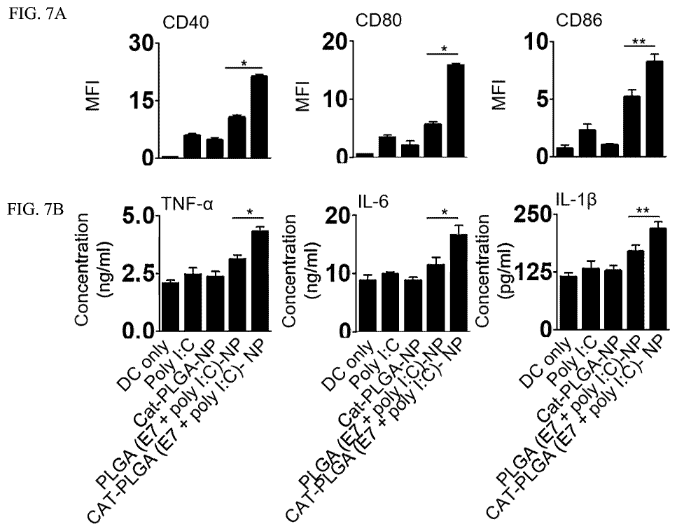

Cell surface markers of DCs were measured using flow cytometry. Compared with other groups, in CAT-PLGA (E7+poly I:C)-NP-treated DCs, it showed that CD40, CD80, and CD86 significantly increased (FIG. 7A). Particularly, significant differences between a control, a poly I:C group, and a CAT-PLGA-NP group were confirmed. In addition, pro-inflammatory cytokines (TNF-α, IL-6, and IL-1β) significantly increased compared to the other groups (FIG. 7B).

### Experimental Result 6: Adhesion of CAT-PLGA-Nps to Mouse Oral Mucosa

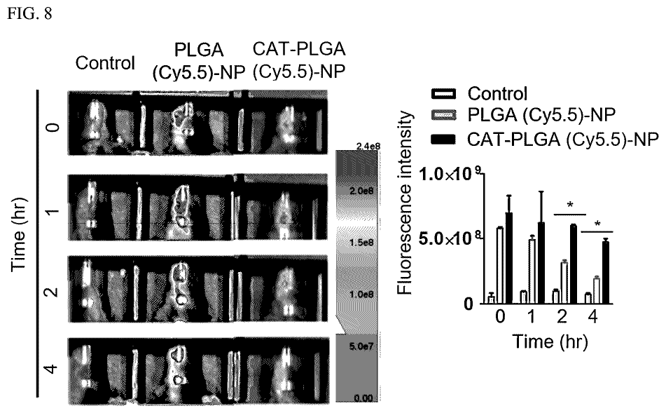

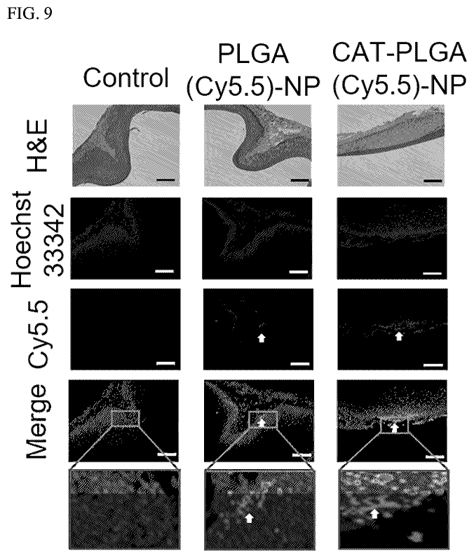

To confirm the adhesion of CAT-PLGA-NPs to an oral mucosal layer, CAT-PLGA(Cy5.5)-NPs were sprayed on the oral mucosal layers of C57BL/6 mice, and fluorescence intensity was confirmed through IVIS. The fluorescence intensity of CAT-PLGA(Cy5.5)-NPs on the mucosal layer was maintained for 4 hours longer than that of PLGA(Cy5.5)-NPs (FIG. 8). In addition, after separating the oral mucosal layers from the C57BL/6 mice, CAT-PLGA(Cy5.5)-NPs were sprayed on the mucosal layers. As a result of confirming the adhesive properties of CAT-PLGA(Cy5.5)-NPs using a fluorescent microscope, the adhesive effect of CAT-PLGA(Cy5.5)-NPs was better than that of PLGA(Cy5.5)-NPs (FIG. 9).

### Experimental Result 7: Therapeutic Efficacy of CAT-PLGA-Nps

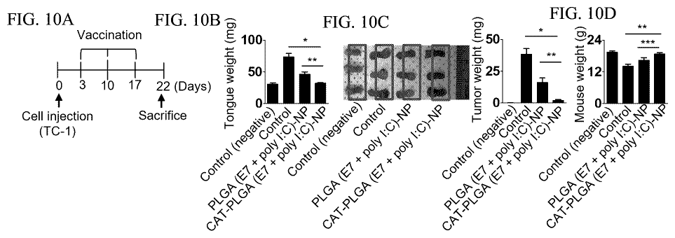

TC-1 cells expressing HPV 16 E6 and E7 proteins are generally used as a tumor model for cancer immunotherapy. Therefore, to measure the therapeutic efficacy of CAT-PLGA-NPs, TC-1 tongue tumor models were developed. To prepare the mouse tongue tumor models, TC-1 cells (4×10⁴ cells/mouse) were injected into the tongues of C57BL/6 mice (n=5 for each group). Vaccination was initiated 3 days after tumor cell injection into the C57BL/6 mice: (1) a control (negative) (2) a control, (3) a PLGA (E7+poly I:C)-NP group, and (4) a CAT-PLGA (E7+poly I:C)-NP group (FIG. 10A). The CAT-PLGA (E7+poly I:C)-NP group exhibited significant suppression of tumor growth compared to the control (56%, p<0.001) and the PLGA (E7+poly I:C)-NP group (30%, p<0.01) (FIGS. 10B and 10C). However, when comparing the CAT-PLGA (E7+poly I:C)-NP group with the control (negative), there were small differences in tongue weight and tumor weight (FIGS. 10B and 10C).

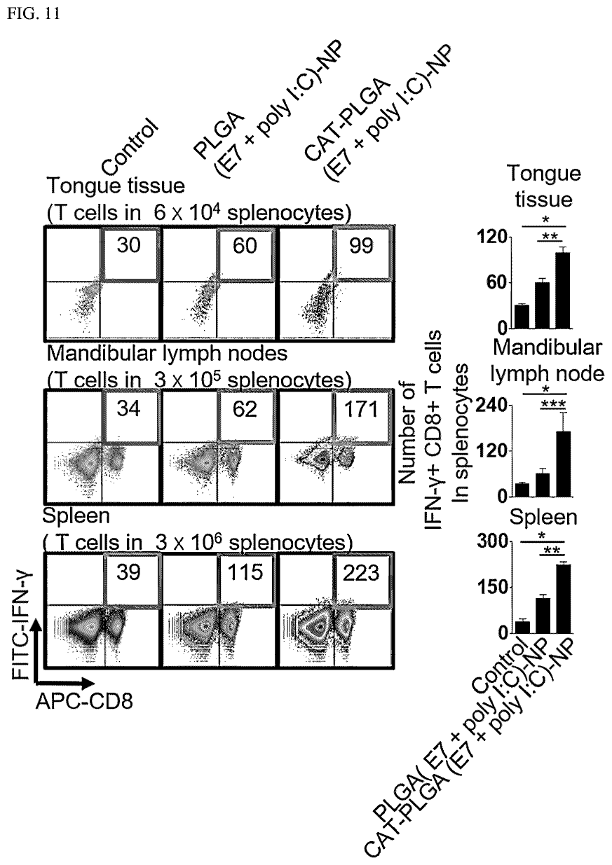

Based on the above results, the body weights of the vaccinated groups were confirmed (FIG. 10D). While CAT-PLGA (E7+poly I:C)-NPs showed consistency, the control (p<0.01) and the PLGA (E7+poly I:C)-NP group (p<0.05) showed a decrease in body weight. This is because the mice in the control and the PLGA (E7+poly I:C)-NP group had difficulty eating food due to tumor growth on the tongue (FIG. 10D). To evaluate CD8+ T cell activation, from the tongue, IFN-γ+CD8+ T cell populations were identified in the tongue, mandibular lymph nodes, and spleen cells. The CAT-PLGA (E7+ poly I:C)-NP group showed a significant increase in the number of IFN-γ+CD8+ T cells compared to those of the control and the PLGA (E7+poly I:C) group, specifically as follows: the control (p<0.001) and the PLGA (E7+poly I:C)-NPs (p<0.01) in the tongue tissue, the control (p<0.001) and the PLGA (E7+poly I:C)-NPs (p<0.05) in the mandibular lymph node, and the control (p<0.001) and the PLGA (E7+poly I:C)-NPs (p<0.01) in the spleen (FIG. 11).

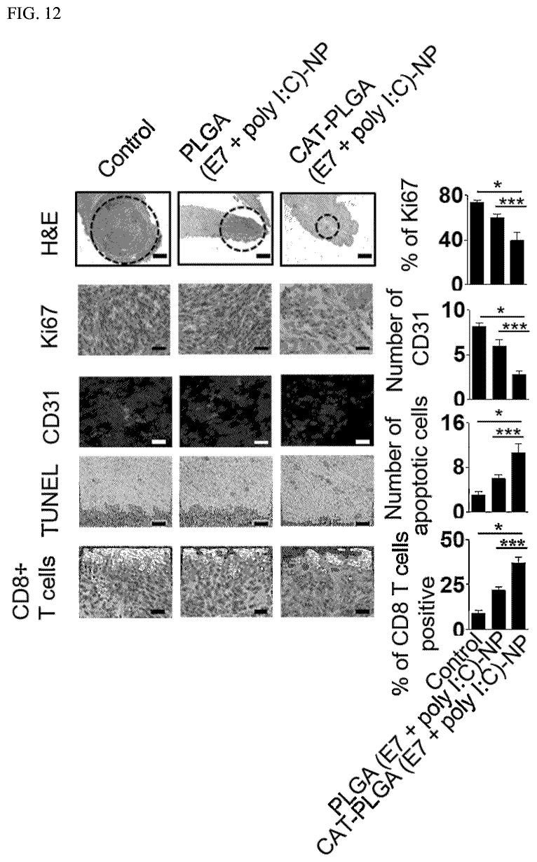

In the control and the PLGA (E7+poly I:C)-NPs, compared to the CAT-PLGA (E7+poly I:C)-NPs, H&E staining analysis showed that a tumor occupied a larger portion of the tongue tissue (FIG. 12). In addition, IHC analysis was used to analyze tumors for markers of cell proliferation (ki67), microvessel density (MVD, CD31), apoptosis (TUNEL), and CD8+ T cells. In the CAT-PLGA (E7+poly I:C)-NPs, compared to the control (p<0.01) and the PLGA (E7+poly I:C)-NPs (p<0.05), the significant suppression of cell proliferation, a decrease in microvessel density, and an increase in apoptotic cells were shown (FIG. 12).

In addition, the number of CD8+ T cells in the tongue tissue of the CAT-PLGA (E7+poly I:C)-NP group was increased compared to those of the control (p<0.001) and the PLGA (E7+poly I:C)-NP group (p<0.01) (FIG. 12).

In the present specification, the detailed description of the content that can be sufficiently recognized and inferred by those of ordinary skill in the art of the present invention was omitted, and other than the specific examples described in the specification, the present invention can be modified in various ways without changing the technical idea or essential structure of the present invention. Accordingly, the present invention can be implemented in ways other than those specifically described and exemplified in the specification, which can be understood by those of ordinary skill in the art of the present invention.

What is claimed is:

1. Mucoadhesive-PLGA nanoparticles in which a mucoadhesive polymer is bound to a surface of poly (D,L-lactide-co-glycolide) (PLGA) nanoparticles.

2. The mucoadhesive-PLGA nanoparticles of claim 1, wherein the mucoadhesive polymer is selected from the group consisting of catechol (CAT), carrageenan, gelatin, pectin, and polyethylene glycol (PEG).

3. The mucoadhesive-PLGA nanoparticles of claim 1, wherein the mucoadhesive polymer is CAT.

4. The mucoadhesive-PLGA nanoparticles of claim 1, wherein the nanoparticles contain an antigen therein.

5. The mucoadhesive-PLGA nanoparticles of claim 4, wherein the antigen is selected from the group consisting of a peptide, siRNA, and mRNA.

6. The mucoadhesive-PLGA nanoparticles of claim 4, wherein the nanoparticles further contain an adjuvant therein.

7. A composition for inducing maturation of antigen-presenting cells (APCs), comprising the mucoadhesive-PLGA nanoparticles of claim 1 as an active ingredient.

8. The composition of claim 7, wherein the APCs are dendritic cells.

9. A pharmaceutical composition for preventing or treating an infectious disease, comprising the mucoadhesive-PLGA nanoparticles of claim 1 as an active ingredient.

10. The pharmaceutical composition of claim 9, which is for spraying into an oral cavity.

11. A pharmaceutical composition for preventing or treating cancer, comprising the mucoadhesive-PLGA nanoparticles of claim 1 as an active ingredient.

12. The pharmaceutical composition of claim 11, which is for spraying into an oral cavity.

13. A method of preparing mucoadhesive-PLGA nanoparticles in which a mucoadhesive polymer is bound to a surface of poly(D,L-lactide-co-glycolide) (PLGA) nanoparticles, comprising:
a) mixing an aqueous solution including an antigen and an adjuvant with an organic solution including PLGA; and
b) mixing the mixture with a compound in which PVA-NH₂ in which an amino group is introduced into polyvinyl alcohol (PVA), and a mucoadhesive polymer in which a carboxyl group is introduced are chemically bonded to each other.

14. A method of preparing CAT-PLGA nanoparticles in which catechol (CAT) is bound to a surface of poly(D,L-lactide-co-glycolide) (PLGA) nanoparticles, comprising:
a) mixing an aqueous solution including an antigen and an adjuvant with an organic solution including PLGA; and
b) mixing the mixture with a compound (PVA-CAT) in which PVA-NH₂ in which an amino group is introduced into polyvinyl alcohol (PVA), and 3,4-dihyroxyhydrocinnamic acid (CAT-COOH) are chemically bonded to each other.
